(19) United States
(12) Patent Application Publication
(10) Pub. No.: US 2008/0274459 A1
(43) Pub. Date: Nov. 6, 2008

(54) METHOD FOR COLLECTING AND STORING BIOLOGICAL MATERIALS

(75) Inventors: Javier Zylberberg, Delray Beach, FL (US); Claudia Zylberberg, Delray Beach, FL (US)

Correspondence Address: AKERMAN SENTERFITT, P.O. BOX 3188, WEST PALM BEACH, FL, 33402-3188, US

(73) Assignee: AssureImmune, LLC, Boca Raton, FL (US)

(21) Appl. No.: 11/743,518

(22) Filed: May 2, 2007

(51) Int. Cl. C12Q 1/68

(52) U.S. Cl. 435/6

(57) ABSTRACT

A method for collecting and storing biological material includes the steps of obtaining a patient identity of a patient and a biological material from a patient. A DNA identity analysis is performed to establish a DNA identity for the patient. The DNA identity is associated with the patient identity and the biological material. The biological material is stored in a preserving environment, and the patient identity and the DNA identity are stored in a database. A request for the biological material is received from a requestor purporting to have the patient identity. A DNA sample from said requestor is obtained. A DNA identity analysis is performed on the DNA sample from the requestor to obtain a DNA identity of the requestor. The DNA identity of the requestor is then compared to the stored DNA identity of the patient, and if the DNA identities match, the biological material is provided to the requestor.

### STATEMENT REGARDING FEDERALLY SPONSORED RESEARCH OR DEVELOPMENT

Not applicable.

### FIELD OF THE INVENTION

This invention relates generally to a method for collecting and storing biological materials.

### BACKGROUND OF THE INVENTION

As the body ages so does its ability to respond to and eliminate pathogens, leading to increased incidence and severity of diseases such as cancers, autoimmune and infectious diseases. At the same time, the efficacy of vaccinations is frequently reduced in older patients as well since the immune system becomes deficient in cells to help the immune response. There exists rapidly developing adoptive immunotherapies to fight cancers, autoimmune conditions, other immunological disorders, cellular immunity, autologous tissue engineering and/or infectious diseases. The collection, testing and preserving of a specific cell supply for use in these therapies is needed. There is a need for the simple, safe, and high quality collection and storage of autologous human white blood cells (WBC) such as monocytes, adult stem cells, tumor cells, tissues, cord blood stem cells and if permitted, embryonic stem cells. The objective is to supply autologous “young” cells, which have a unique biological quality with high potential preventative and curative clinical impact, to patients at a future time when they are fighting a disease requiring healthy and safe cells, such as WBCs, to re-build their immune system or repair specific areas. Cord blood has proven to be an excellent therapeutic source of stem cells that have the ability to enhance a person's blood producing capability and immune system. To date some 50 disorders can be treated with stem cells sourced from cord blood. This is an area that is quickly being developed and holds great promise in the future of regenerative medicine. Procedures for the collection and storage of cord blood stem cells are also needed.

### SUMMARY OF THE INVENTION

A method for collecting and storing biological material includes the step of obtaining a patient identity of a patient. Biological material is obtained from the patient. DNA identity analysis is performed to establish a DNA identity for the patient. The DNA identity is associated with the patient identity and the biological material. The biological sample is stored in a preserving environment, and the patient identity and the DNA identity are stored in a database.

A DNA sample is obtained from a requestor purporting to have the patient identity. A DNA identity analysis is performed on a DNA sample from the requestor to obtain a DNA identity of the requestor. The stored DNA identity of said patient is compared with the DNA identity of the requestor. If the DNA identities match, the biological material is produced to the requestor.

The biological material can be stem cells, such as hematopoietic stem cells from peripheral blood, hematopoietic stem cells from bone marrow, mesenchymal stem cells, and cord blood stem cells. the biological material can be white blood cells (WBC) such as monocytes and neutrophils. Other biological materials, such as tumor cells, can be stored according to the invention.

The DNA identity analysis can be performed by any suitable method, such as PCR analysis. Other DNA identity analysis techniques can be used.

The storage of the biological materials can include the step of dividing the biological material into portions and storing the divided material portions in different physical locations. The storing step cam be a suitable method such as cryogenic storage of the biological material.

The step of obtaining of the biological material can include the provision of a kit for this purpose. The kit can include a sealable container, one or more sealable test tubes, and/or a syringe for collecting the material. The obtaining of the biological material can include sending a mobile unit to obtain the biological material.

### BRIEF DESCRIPTION OF THE DRAWINGS

There are shown in the drawings embodiments that are presently preferred it being understood, however, that the invention is not limited to the precise arrangements and instrumentalities shown, wherein:

FIG. 1 is a schematic diagram illustrating a method for collecting and storing biological materials according to the invention.

### DETAILED DESCRIPTION OF THE PREFERRED EMBODIMENTS

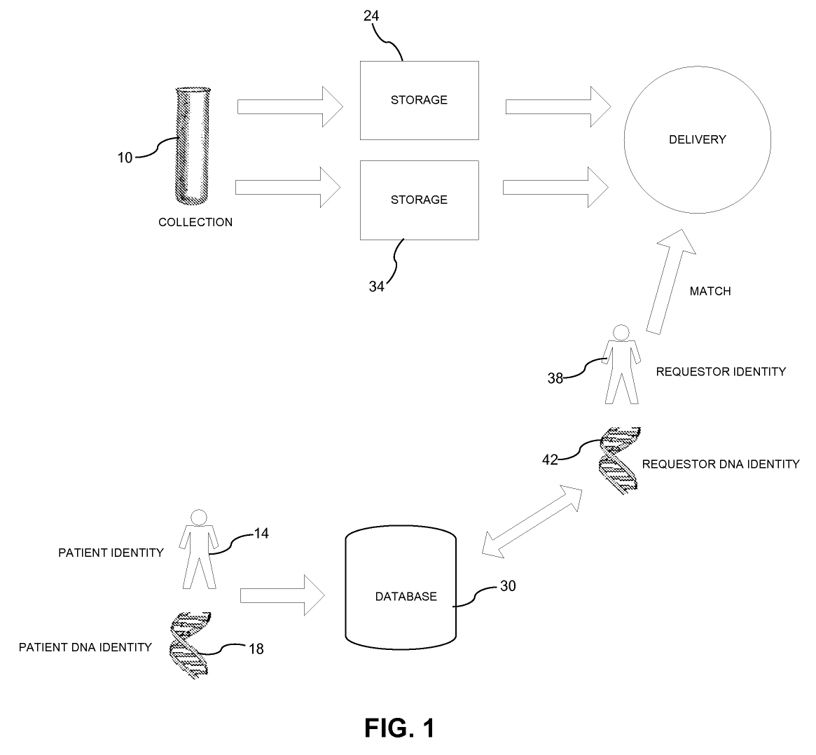

The collection and storage of biological samples, particularly for personalized use, requires that the samples be collected safely and securely, and then stored in a preserving environment until they are needed. There is shown in FIG. 1 a schematic diagram depicting the process. The biological material 10 is collected from the patient. An identity of the patient 14 is obtained. A DNA identity analysis is performed to establish a DNA identity 18 of the patient. The DNA identity can be established with a biological sample from the patient that is different from the biological material that is to be stored, or it can be established from the same biological material. The biological material 10 is then stored in a preserving environment 24, and the patient identity and the DNA identity are then stored in a database 36. The biological material can be stored in more than one physical location, such as secondary location 34 in order to provide redundancy and security that the biological material will be available when needed. At a later date when a person requests delivery of the biological material, the requestor must provide a register identity 38 and provide a DNA sample from which the requestor's DNA identity 42 can be established. If the requestor's patient identity and DNA identity matches the stored patient identity and DNA identity, the requestor will be provided the biological material.

The biological sample can be collected in any suitable fashion, and by any suitable means. The manner of collection will in part depend upon the biological material that is being collected. A kit can be provided at the collection site to facilitate the collection process, and to provide ready implements to medical staff who otherwise may not collect the biological material. For example, in the case of cord blood stem cells, the kit can include a syringe suitable for removing blood from the umbilical cord that has been tied and clamped after delivery. A sealable container can be provided for purposes of transporting the blood to a storage facility. Test tubes for sending test samples to laboratories can be provided. In some cases, special purpose collection facilities may be provided. The collection facilities can have suitable accreditations, such as FDA-HCT/P registered, ISO Certified 9001:2000, cGMP/cGTP compliant, AABB accredited. The collection may be performed during a routine blood donation or as part of other routine medical procedures, or non-routine procedures such as surgeries. Mobile units can be provided to facilitate the collection process, or collection and transportation to a storage site.

The types of cells that are collected will vary according to the intended therapy, whether the cells are necessary for immunotherapies to fight cancers, autoimmune conditions, other immunological disorders, cellular immunity, autologous tissue engineering and/or infectious diseases. WBCs (monocytes) and stem cells, particularly cord blood stem cells and adult stem cells from peripheral blood, are of current interest, although other types of cells and tissues can be collected and stored according to the methods of the invention. Tumor cells may be collected for purposes of storage for purposes of creating therapies which specifically target the tumor. Embryonic stem cells can be collected if permitted.

The identity of the patient can be registered with a variety of different information. Such information can include name, address, date of birth, Social Security Number or the like, or any other indicia useful to identify the patient. Also, one or more passwords, codes or other confidential identification can be assigned or created by the patient for use in uniquely identifying the patient. Biometric indicia such as fingerprints, retinal scans, and voice identification can also be used. The patient identity is stored in a suitable secure database 30, or in multiple databases to provide security and redundancy.

The biological material 10 can be stored in a suitable storage facility 24 according to a suitable protocol for the particular biological material that is being stored. The sample must be stored in a preserving environment. Such preservation is usually accomplished by cryogenic storage, however, other preservation methods such as culturing are also possible. The cells collected will be cryo-preserved immediately after collection and shipped to secure storage locations. The storage locations will have suitable facilities for storing the particular cells that are of interest. Appropriate regulatory protocols for such storage facilities are sometimes in place and must be followed. The biological material can be sent to more than one storage facility around the globe, such as secondary storage facility 34, to ensure security and redundancy within the system.

The request for the biological material, or any portion of the biological material can be received by any suitable means. The requestor must provide a requestor identity 38 which matches that of the patient by providing the appropriate identifying material, such as name, address, date of birth, Social Security number, or the like, or possibly also by appropriate biometric means such as fingerprint, retinal scan, or voice identification. The requestor must also send a DNA sample from which a DNA identity 42 of the requestor can be obtained. DNA identity testing procedures performed at the time of collection and again at the time of cell retrieval from the bank ensure an accurate ownership match.

On line web access can be provided such that the patient or a doctor or representative of the patient can monitor the location and status of the biological sample. Appropriate login and security measure such as password protection and encryption can be provided. Payment information and systems can also be provided to ensure up to date and accurate accounts.

The web site account number can inform the patient of various account information. Such information can include, but is not limited to, the number of cells in storage, their location and date of deposit. Other information can include test results from testing such as HLA, DNA and infectious diseases.

This invention can be embodied in other forms without departing from the spirit or essential attributes thereof, and accordingly, reference should be had to the following claims as indicating the scope of the invention.

We claim:

1. A method for collecting and storing biological material, comprising the steps of:
obtaining a patient identity of a patient;
obtaining a biological material from said patient;
performing DNA identity analysis to establish a DNA identity for said patient;
associating said DNA identity with said patient identity and said biological material;
storing said biological sample in a preserving environment, and storing said patient identity and said DNA identity in a database; and,
obtaining a DNA sample from a requestor purporting to have said patient identity, performing a DNA identity analysis on a DNA sample from said requestor to obtain a DNA identity of said requester, and comparing said stored DNA identity of said patient with said DNA identity of said requester, and if said DNA identities match, producing at least a portion of said biological material to said requester.

2. The method of claim 1, wherein said biological material comprises stem cells.

3. The method of claim 2, wherein said stem cells are cord blood stem cells.

4. The method of claim 1, wherein said biological material comprises white blood cells.

5. The method of claim 1, wherein said DNA identity analysis comprises PCR analysis.

6. The method of claim 1, wherein said storage of said biological materials comprises dividing said biological material into portions and storing said divided material portions in different physical locations.

7. The method of claim 1, wherein said storing step comprises cryogenic storage of said biological material.

8. The method of claim 1, wherein said obtaining of said biological material comprises providing a kit, said kit comprising a sealable container and a syringe.

9. The method of claim 1, wherein said obtaining of said biological material comprises sending a mobile unit to obtain said biological material.

10. The method of claim 1, wherein said biological material comprises tumor cells.
